(19) United States
(12) Patent Application Publication
(10) Pub. No.: US 2012/0329131 A1
(43) Pub. Date: Dec. 27, 2012

(54) METHOD FOR INCREASING THE ACTIVITY OF SUPEROXIDE DISMUTASE

(76) Inventors: Pao-Chuan HSIEH, Pingtung (TW); Wan-Ting Su, Tainan City (TW)

(21) Appl. No.: 13/166,402

(22) Filed: Jun. 22, 2011

(51) Int. Cl. C12N 9/02

(52) U.S. Cl. 435/189

(57) ABSTRACT

A method for increasing the activity of superoxide dismutase comprises steps of: liquid culture, by providing a strain of Bacillus subtilis and a liquid medium to carry out a fed-batch fermentation and to obtain a primary fermentation broth, wherein the liquid medium has 8 wt % to 10 wt % of maltose, 10 wt % to 15 wt % of soya powder and rest of water; and solid culture, by culturing the primary fermentation broth in a solid medium, which has solid substrate and maltose in a ratio of 10:1 to 20:1, to obtain a secondary fermentation broth with SOD in high activity, wherein the solid substrate contains wheat germ and water.

### BACKGROUND OF THE INVENTION

1. Field of the Invention

The present invention relates to a method for increasing the activity of superoxide dismutase (SOD) and, more particularly, to a method for increasing the activity of SOD which is produced by Bacillus subtilis.

2. Description of the Related Art

Reactive oxygen species (ROS), being natural byproducts of the normal metabolism of oxygen in organisms, plays an important role in cell signaling and homeostasis. Generally, the ROS will damage to cells, leading to DNA mutation and cell toxication. Accordingly, cells are able to develop a defense system to against ROS damage with enzymes, for example, superoxide dismutases (SOD) and catalases. With this circumstance, intracellular ROS are sufficiently removed in preventing from damages caused by free radicals.

Currently, SOD has been widely put to use in pharmaceutics, in order to manufacture into health products, cosmetics, or medication for improving autoimmune diseases, endocrine diseases, inflammation, and aging. Furthermore, SOD can also be used in food-processing industry, as a food additive to prolong the storage life of food products.

In conventional arts, SOD is generally obtained from liver cells, erythrocyte, garlic, corn germ or cactus via a method of extraction, chromatography, or salting-out, and however, the production, as well as the activity of SOD is poor and low.

Referring to Taiwan Publication No. 200741006, entitled “a method for producing highly active SOD and its use in solid and liquid fermentation,” a conventional method for producing SOD is disclosed by using Bacillus subtilis to produce SOD via a liquid fermentation or a solid fermentation.

In the liquid fermentation of the conventional method, a liquid medium, being pH6.0 to 7.5, is provided and comprises at least two carbon sources and nitrogen sources, wherein the carbon sources of the liquid medium are selected from a group of glucose, sorbitol and soluble starch, and the nitrogen sources of the liquid medium are selected form a group of soya powder, beef extraction and peptone.

In the solid fermentation of the conventional method, a solid medium is provided and comprises a solid fermentation substrate, comprising wheat germ and soya powder, and carbon source, wherein the carbon source is selected from a group of glucose, sorbitol, and soluble starch.

In conventional art, whatever in liquid fermentation or solid fermentation, multiplicate carbon sources and nitrogen sources are needed to maintain the growth of Bacillus subtilis, and also to sustain the production of SOD in the Bacillus subtilis. Accordingly, the cost of the conventional method for producing SOD is high. Furthermore, the activity of SOD obtained from the conventional method is around 21000 U/ml, which may be limited in used.

Hence, there is a need for providing a manufacturing method of print circuit board so as to improve the above disadvantages of the conventional method, and to obtain SOD in high activity.

### SUMMARY OF THE INVENTION

The primary objective of this invention is to provide a method for increasing the activity of superoxide dismutase, in which a liquid medium and a solid medium are sequentially used to culture bacteria so as to promote the activity of superoxide dismutase in products.

The secondary objective of this invention is to provide a method for increasing the activity of superoxide dismutase, in which a single carbon source and single nitrogen source is used both in the liquid medium and the solid medium to culture Bacillus subtilis, so that superoxide dismutase in high activity can be produced by the Bacillus subtilis in a cost-saving process.

A method for increasing the activity of superoxide dismutase comprises steps of: liquid culture, by providing a strain of Bacillus subtilis and a liquid medium to carry out a fed-batch fermentation and to obtain a primary fermentation broth, wherein the liquid medium has 8 wt % to 10 wt % of maltose, 10 wt % to 15 wt % of soya powder and rest of water; and solid culture, by culturing the primary fermentation broth in a solid medium, which has solid substrate and maltose in a ratio of 10:1 to 20:1, to obtain a secondary fermentation broth with SOD in high activity, wherein the solid substrate contains wheat germ and water.

Further scope of the applicability of the present invention will become apparent from the detailed description given hereinafter. However, it should be understood that the detailed description and specific examples, while indicating preferable embodiments of the invention, are given by way of illustration only, since various others will become apparent from this detailed description to those skilled in the art.

### BRIEF DESCRIPTION OF THE DRAWINGS

The present invention will become more fully understood from the detailed description given herein below and the accompanying drawings which are given by way of illustration only, and thus are not limitative of the present invention, and wherein:

FIG. 1 is a diagram illustrating a method for increasing the activity of superoxide dismutase in the present invention;

FIG. 2 is a bar chart illustrating the activity of SOD of groups A0 to A13 in the present invention;

FIG. 3 is a bar chart illustrating the activity of SOD of groups B1 to B14 in the present invention;

FIG. 4 is a bar chart illustrating the activity of SOD of groups C1 to C8 in the present invention;

FIG. 5 is a bar chart illustrating the activity of SOD of groups D1 to D8 in the present invention;

FIG. 6 is a line chart illustrating the activity of SOD of groups E1 to E10 in the present invention;

FIG. 7 is a line chart illustrating the activity of SOD of groups F1 to F5 in the present invention;

FIG. 8 is a line chart illustrating the pH of liquid medium in the present invention;

FIG. 9 is a line chart illustrating the pH of liquid medium, the activity of SOD, and the bacteria count of Bacillus subtilis in the present invention;

FIG. 10 is a bar chart illustrating the activity of SOD of groups H1 to H3 in the present invention.

All figures are drawn for ease of explaining the basic teachings of the present invention only; the extensions of the figures with respect to number, position, relationship, and dimensions of the parts to form the preferred embodiment will be explained or will be within the skill of the art after the following teachings of the present invention have been read and understood. Further, the exact dimensions and dimensional proportions conforming to specific force, weight, strength, and similar requirements will likewise be within the skill of the art after the following teachings of the present invention have been read and understood.

### DETAILED DESCRIPTION OF THE INVENTION

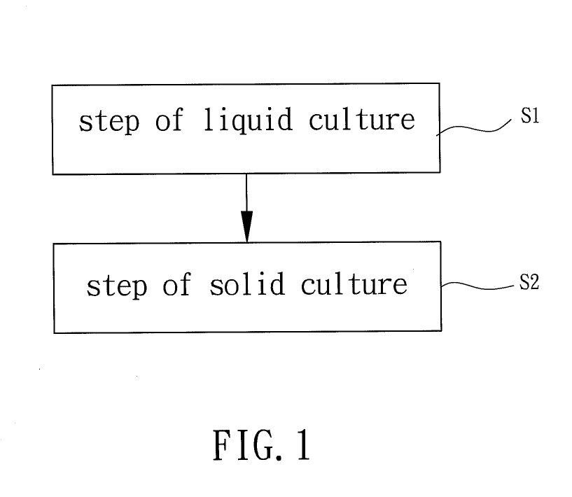

With reference to FIG. 1, the present invention provides a method for increasing the activity of superoxide dismutase (SOD), which comprises a step of “liquid culture S1,” and a step of “solid culture S2”.

In the step of “liquid culture S1,” a strain of Bacillus subtilis is prepared and carried out fed-batch fermentation in a liquid medium at 37±2° C. for 48±2 hours, in order to obtain a primary fermentation broth. In the present invention the weight ratio between the Bacillus subtilis and the liquid medium is 95:5 to 97:3.

More precisely, the fed-batch fermentation of the present invention can be single fed-batch fermentation or repeated fed-batch fermentation, with feeding a solution of the limiting substrate at the same concentration, for example fresh liquid medium, according to the pH of the liquid medium during the fermentation. In the present invention, the pH of the liquid medium is preferably to be maintained at pH5.5±2 at lowest. Furthermore, the volume ratio of the limiting substrate provided during the fermentation is around 40% to 60% in the liquid medium, so that, the conditions in the provision of the liquid medium during the fermentation can be successfully controlled, in order to meet the requirement of the growth of Bacillus subtilis. In the present embodiment, fresh liquid medium is additionally added at the 8^{th} and 26^{th} hours of the fermentation, and the primary fermentation broth is obtained at the 48±2^{th} hours of the fermentation.

In the present embodiment, the liquid medium has 8 wt % to 10 wt % of maltose, being the single carbon source of the liquid medium, 10 wt % to 15 wt % of soya powder, being the single nitrogen source of the liquid medium, and the remaining percent of water, preferably the liquid medium can further has other nutrients or microelements, such as sodium chloride, preferable at a concentration of 0.1%, tryptophan, preferable at a concentration of 0.5, vitamin B groups or vitamin B12, preferable at a concentration of 10 ppm, for the sake of further adjusting the conditions of the liquid medium, for example osmotic pressure, to meet to the requirement of the growth of the Bacillus subtilis. In this way, the Bacillus subtilis can be grown in the liquid medium in a high efficiency.

In the step of “solid culture S2,” the primary fermentation broth obtained from the step of “liquid culture S1” is further cultured in a solid medium at 30±2° C., for 72±2 hours, in order to obtain a secondary fermentation broth contained highly active SOD, wherein the ratio between the primary fermentation broth and the solid medium is 5:95 to 15:85. More precisely, the concentration of the primary fermentation broth is no less than 1×10⁹ cfu/ml, with the primary fermentation broth being attached at the solid medium, and with the carbon source and nutrients of the wheat germ in the solid medium to promote the growth of the Bacillus subtilis on the solid medium, so as to obtain the secondary fermentation broth with highly active of SOD, for example more than 25000 U/mg in SOD activity.

In the present embodiment, the solid medium is prepared by steaming the wheat germ at 100±2° C. for 20 to 40 minutes, combining the steamed wheat germ with water in a ratio of 100:45 to 100:75 to obtain a solid substrate, and then mixing up the solid substrate with 0.1 to 0.2 weight times of the maltose. Generally, wheat germ takes 25% weight of whole wheat and is rich in 12% of oil, particular to unsatisfied fatty acid, 8% of protein, 7% of pantothenic acid and various microelements. With the above arrangement, the wheat germ in the solid medium will be loose, soft, and water-absorbent, so that the nutrients of the wheat germ are easy to be used by the Bacillus subtilis. Furthermore, the solid medium contains approximately 45% to 75%, particularly to 60%, of water, which can facilitate the absorption of nutrients of the wheat germ in the Bacillus subtilis and advance the production of highly active SOD.

In the present invention, the Bacillus subtilis is cultured at a liquid medium at first to obtain a primary fermentation broth, followed by further culturing the primary fermentation broth at a solid medium to obtain a secondary fermentation broth and highly active SOD therein. The liquid medium of the present invention contains single carbon source and single nitrogen source, and therefore, the preparation of the liquid medium can be achieved in a simple and cost-saving process. Moreover, the single carbon source of the present is selected as maltose, and the single nitrogen source of the present invention is selected as soya powder, with the result to promote the growth of the Bacillus subtilis and also to increase the activity of SOD produce by the Bacillus subtilis.

In order to prove the advantages of the method for increasing the activity of SOD in the present invention, a strain of Bacillus subtilis is prepared and used in diverse trials, including (A) different carbon sources; (B) different nitrogen sources; (C) different amino acid; (D); different microelements; (E) different conditions of solid substrate; and (F) different carbon sources of solid mediums, in which the Bacillus subtilis is separately cultured under various conditions in each trial to obtain their primary or secondary fermentation broth, and then the activity of SOD in each primary or secondary fermentation broth is demonstrated respectively. In accordance with TABLE 1, the Bacillus subtilis is grown in a nutrient broth at 37±2° C., for 18±2 hours before the trails (A) to (F) to obtain a bacterial broth, which is ready to be used in the trials (A) to (F). Furthermore, the Bacillus subtilis of the present embodiment is purchased from Bioresource Collection and Research Center in Taiwan, and however, other strains of Bacillus subtilis whatever purchased from any institutions or newly isolated are sufficient to be used in the trials (A) to (F) of the present invention.

TABLE 1
Nutrient Broth

| Contents | Units (g) |
|---|---|
| Tryptone | 10 |
| Yeast extract | 5 |
| NaCl | 10 |
| Water | Add till 1 L |

In the present invention, the activity of SOD in each primary or secondary fermentation broth obtained from each trail is analyzed via nitroblue tetrazolium assay (NBT assay), by providing NBT to each primary or secondary fermentation broth, with the SOD therein inhibiting the deoxidization of NBT; quantifying methyl hydrazone with a spectrophotometer under 560 nm; and identifying the activity of the SOD as compared to a control via FORMULA 1. In FORMULA 1, the Trial_{OD560} means the absorbance of trial samples under 560 nm, and the Control_{OD560} means the absorbance of control samples under 560 nm.

SOD activity=(1−Trial_{OD560}/Control_{OD560})÷0.5×100%×dilution FORMULA 1

In the present embodiment, the trail samples are prepared by individually combining 0.1 ml of each primary or secondary fermentation broth with 0.2 ml EDTA, 0.1 ml NBT (2.45 mM) and 3.0 ml phosphate buffer (67 mM, pH7.8) to obtain a mix, followed by keeping the mix at 37° C. for 5 minutes and co-incubating the mix with 50 μl riboflavin (1.2 mM) under illumination of light in 13 W for 30 minutes. Similarly, the control sample is also prepared according to the protocol above, however with the primary or secondary fermentation broth of each trail sample being replaced by the same volume of phosphate buffer.

Referring to TABLE 2, various liquid mediums, including (A0) to (A13), are prepared, followed by inoculating 5% of the bacterial broth into each liquid medium respectively and carrying out a fermentation at 37±2° C., for 48±2 hours. The liquid mediums (A0) to (A13) all comprise 100 g of carbon source and 50 g of soya powder in 1 liter of water, wherein the carbon source in each liquid medium is different. In the present embodiment, primary fermentation broths of (A0) to (A13) are collected and analyzed via the NBT assay.

TABLE 2
Groups Arrangements in the Trial (A)

| Groups | Carbon Sources | SOD Activity (U/ml) |
|---|---|---|
| A0 | — | 898.26 |
| A1 | Maltose | 1568.89 |
| A2 | Fructose | 937.07 |
| A3 | Lactose | 1287.54 |
| A4 | Glucose | 734.38 |
| A5 | Melibiose | 1143.75 |
| A6 | Fructo-oligo saccharides | 778.13 |
| A7 | Galactose | 978.13 |
| A8 | Sucrose | 987.50 |
| A9 | Mannitol | 1408.21 |
| A10 | Sorbose | 336.93 |
| A11 | Mannose | 894.17 |
| A12 | Xylose | 472.05 |
| A13 | Soluble starch | 1387.16 |

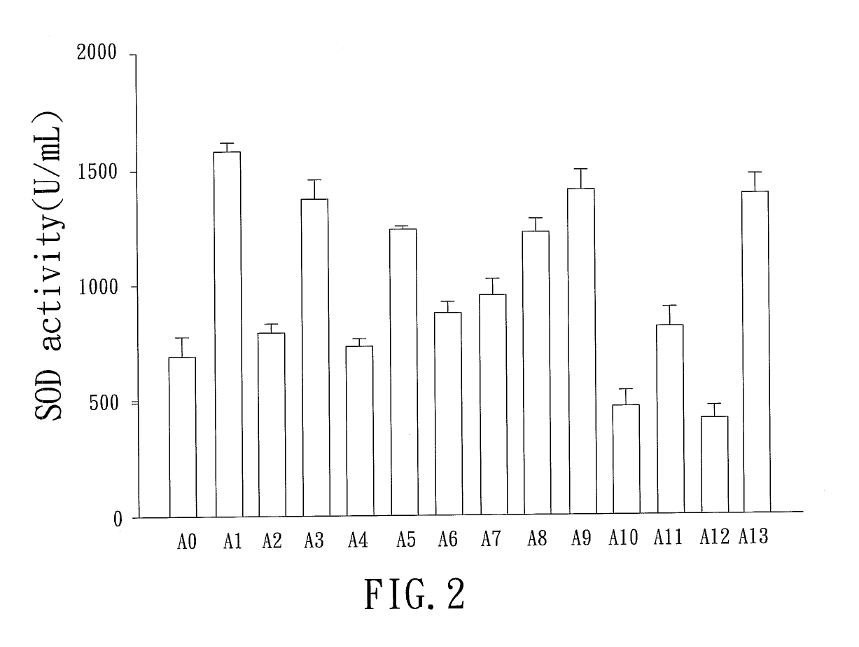

According to TABLE 2 and FIG. 2, it is suggested that, with the performance of maltose in the liquid medium, SOD in high activity can be produce by the Bacillus subtilis.

Referring to TABLE 3, various liquid mediums, including (B 1) to (B 14), are prepared, followed by inoculating 5% of the bacterial broth into each liquid medium respectively and carrying out a fermentation at 37±2° C., for 48±2 hours. The liquid mediums (B1) to (B 14) all comprise 100 g of maltose and 50 g of nitrogen source in 1 liter of water, wherein the nitrogen source in each liquid medium is differ from groups. In the present embodiment, primary fermentation broths of (B 1) to (B 14) are collected and analyzed individually via the NBT assay.

TABLE 3
Groups Arrangements in the Trial (B)

| Groups | Nitrogen Sources | SOD Activity (U/ml) |
|---|---|---|
| B1 | Yeast extract | 1123.34 |
| B2 | Soybean powder | 1558.82 |
| B3 | Tryptone | 1079.20 |
| B4 | Nutrient broth | 945.95 |
| B5 | Soytone | 1209.22 |
| B6 | Peptone | 560.06 |
| B7 | Soypeptone | 1052.41 |
| B8 | Tryptic soy broth | 888.09 |
| B9 | Soya powder + Peptone | 1314.81 |
| B10 | Soya powder + Soytone | 1055.75 |
| B11 | Soya | 1379.70 |
| | powder + Soypeptone | |
| B12 | Soya powder + Yeast | 1131.45 |
| | extract | |
| B13 | Soya powder + Tryptone | 1224.13 |
| B14 | Soya powder + Peptone | 822.46 |

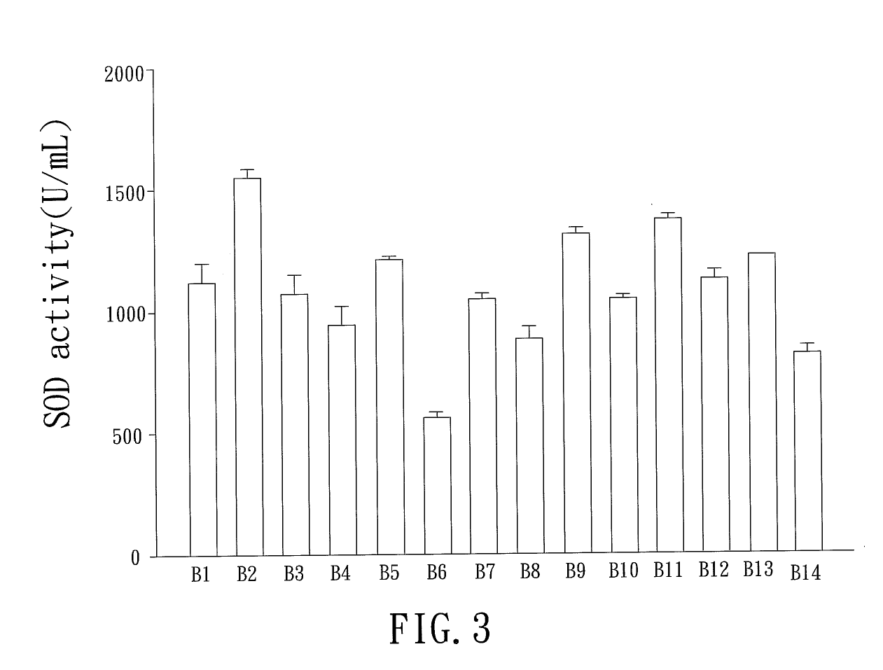

According to TABLE 3 and FIG. 3, it is suggested that, with the performance of soya powder in the liquid medium, SOD in high activity can be produce by the Bacillus subtilis.

Referring to TABLE 4, various liquid mediums, including (C1) to (C8), are prepared, followed by inoculating 5% of the bacterial broth into each liquid medium respectively and carrying out a fermentation at 37±2° C., for 48±2 hours. The liquid mediums (C1) to (C8) all comprise 100 g of maltose, 5.0 g of amino acid and 50 g of soya powder in 1 liter of water, wherein the amino acid in each liquid medium is differ from groups. In the present embodiment, primary fermentation broths of (C1) to (C8) are collected and analyzed individually via the NBT assay.

TABLE 4
Groups Arrangements in the Trial (C)

| Groups | Amino Acids | SOD Activity (U/ml) |
|---|---|---|
| C1 | Glutamic acid | 1402.50 |
| C2 | Tryptophan | 2107.29 |
| C3 | Arginine | 1186.31 |
| C4 | Asparatic acid | 1863.15 |
| C5 | Methionine | 1176.65 |
| C6 | Monosodium glutamate | 1416.74 |
| C7 | Valine | 1766.26 |
| C8 | Leucine | 1388.66 |

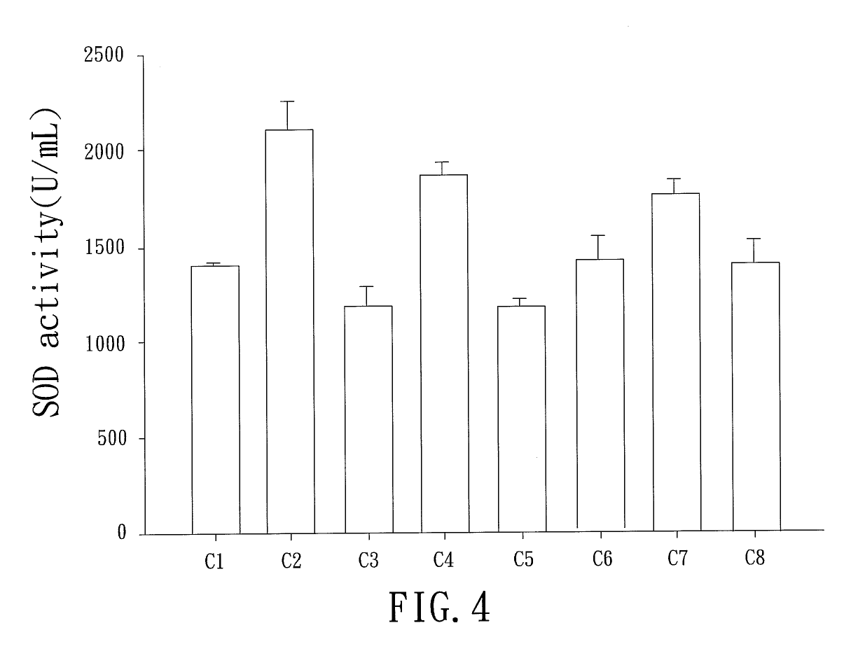

According to TABLE 4 and FIG. 4, it is suggested that, with the performance of tryptophan in the liquid medium, SOD in high activity can be produce by the Bacillus subtilis.

Referring to TABLE 5, various liquid mediums, including (D1) to (D8), are prepared, followed by inoculating 5% of the bacterial broth into each liquid medium respectively and carrying out a fermentation at 37±2° C., for 48±2 hours. The liquid mediums (D1) to (D8) all comprise 100 g of maltose, 5.0 g of tryptophan, 10 ppm of microelement, and 50 g of soya powder in 1 liter of water, wherein the microelement in each liquid medium is differ from groups. In the present embodiment, primary fermentation broths of (D1) to (D8) are collected and analyzed individually via the NBT assay.

TABLE 5
Groups Arrangements in the Trial (D)

| Groups | Microelements | SOD Activity (U/ml) |
|---|---|---|
| D1 | Vitamin C | 1717.03 |
| D2 | Vitamin B1 | 1156.98 |
| D3 | Vitamin B2 | 1825.50 |
| D4 | Vitamin B3 | 1258.99 |
| D5 | Vitamin B6 | 2328.35 |
| D6 | Vitamin B groups | 2318.12 |
| D7 | Biotin | 1539.51 |
| D8 | Vitamin E | 1435.11 |

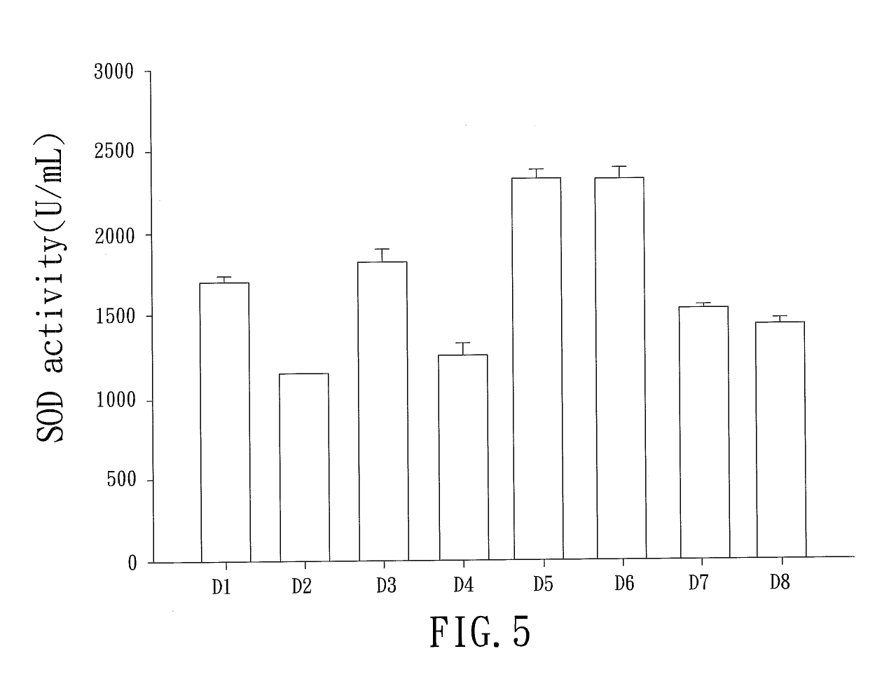

According to TABLE 5 and FIG. 5, it is suggested that, with the performance of vitamin B6 or vitamin B groups in the liquid medium, SOD in high activity can be produce by the Bacillus subtilis.

Referring to TABLE 6, the primary fermentation broth of trial (A1) is further inoculated on various solid mediums, including (E1) to (E10) to carry out a fermentation at 37±2° C., for 48±2 hours, wherein wheat germs in solid mediums (E1) to (E10) are separately mixed up water in 45 wt %, 50 wt %, 55 wt %, 60 wt %, 65 wt %, 70 wt %, 75 wt %, 80 wt %, 85 wt %, and 90 wt %. In the present embodiment, secondary fermentation broths of (E1) to (E10) at a concentration of 10⁹ cfu/ml, are collected and analyzed individually via the NBT assay.

TABLE 6
Groups Arrangements in the Trial (E)

| | Wt % of Water in Solid | SOD Activity |
|---|---|---|
| Groups | Substrate | (U/ml) |
| E1 | 45% | 21632.20 |
| E2 | 50% | 19821.43 |
| E3 | 55% | 17856.75 |
| E4 | 60% | 21822.94 |
| E5 | 65% | 20986.62 |
| E6 | 70% | 19218.22 |
| E7 | 75% | 18302.90 |
| E8 | 80% | 12318.84 |
| E9 | 85% | 20164.44 |
| E10 | 90% | 12687.52 |

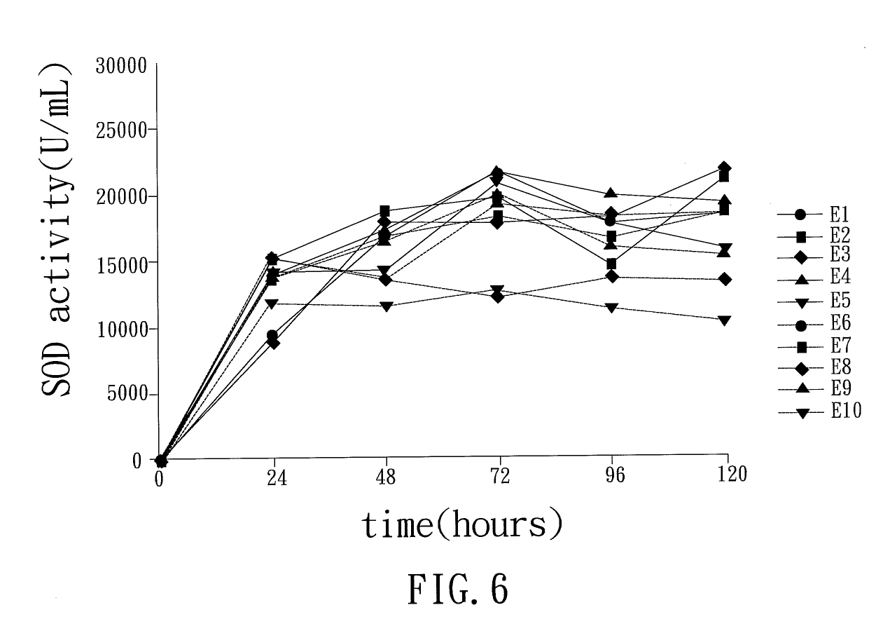

According to TABLE 6 and FIG. 6, it is suggested that, SOD in high activity can be obtained from the Bacillus subtilis, which is grown on solid mediums contained 45% to 75%, particularly to 55% to 65% of water in solid substrate.

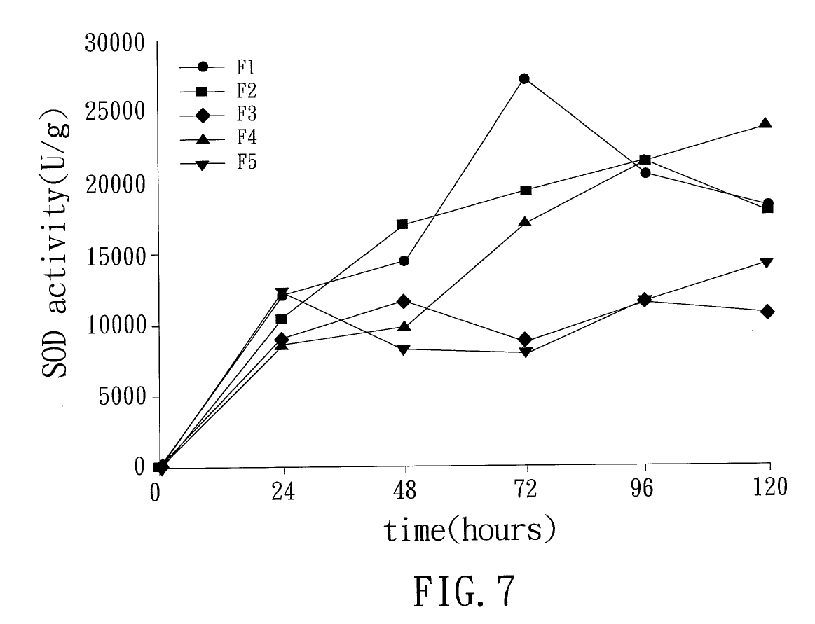

Referring to TABLE 7 and FIG. 7, the primary fermentation broth of trial (A1) is further inoculated on various solid mediums, including (F1) to (F5) to carry out a fermentation at 37±2° C., for 48±2 hours, wherein carbon sources in solid mediums (F1) to (F5) are all diverse. In the present embodiment, secondary fermentation broths of (F1) to (F5) are collected and analyzed individually via the NBT assay.

TABLE 7
Groups Arrangements in the Trial (F)

| Groups | Carbon sources | SOD Activity (U/ml) |
|---|---|---|
| F1 | Maltose | 27021.38 |
| F2 | Fructose | 19175.46 |
| F3 | Glucose | 8648.65 |
| F4 | Soluble starch | 16912.91 |
| F5 | Sucrose | 7952.47 |

According to TABLE 7 and FIG. 7, it is suggested that, with the performance of maltose in the solid medium, SOD in high activity can be produce by the Bacillus subtilis.

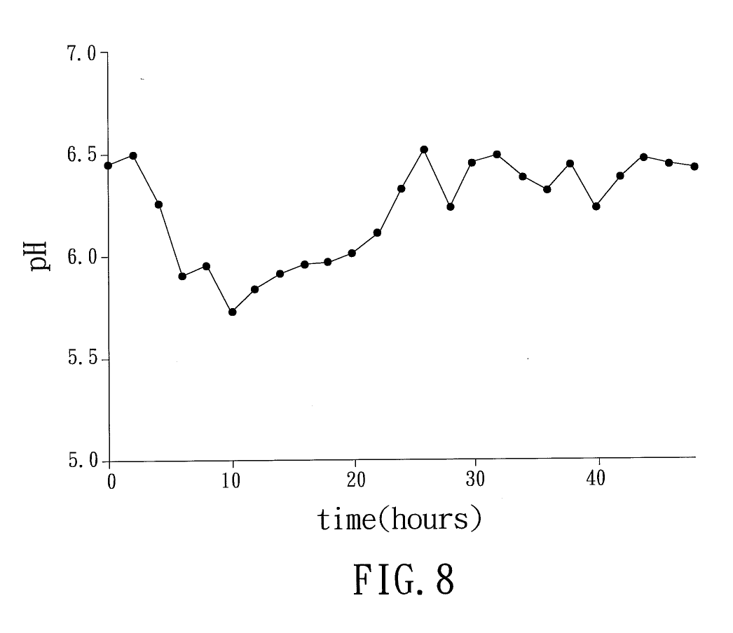

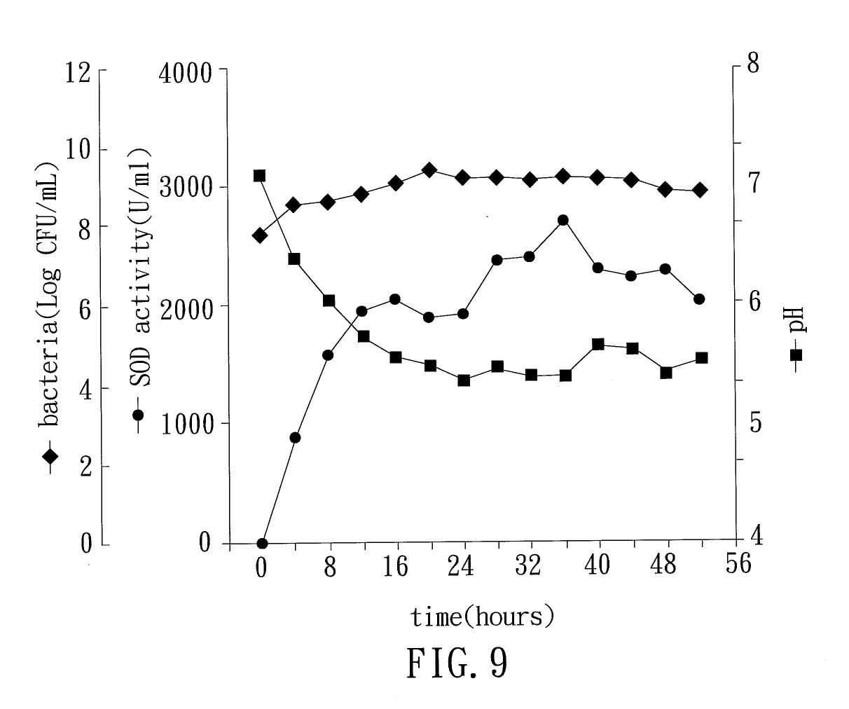

FIG. 8 indicates the pH of the liquid medium during the fermentation. It is noted that due to the accumulation of metabolites produced by the Bacillus subtilis during the fermentation, the pH of the liquid medium will significantly decrease, particularly at the 10^{th} and 26^{th} hours of the fermentation. In contrast, FIG. 9 shows the pH of the liquid medium, bacterial count, and the SOD activity during the fed-batch fermentation of the present invention. It is obvious that, with the performance of the fed-batch fermentation in the step of liquid culture of the present invention, the pH of the liquid medium can be successfully maintained at no less than pH 5.5, so as to promote the activity of SOD produced by the Bacillus subtilis.

Finally, to further verifying the advantages of the method for increasing the activity of SOD in the present invention, the bacterial broth are prepared and assigned to 3 groups, including (H1) co-incubating 5% the bacterial broth and the liquid medium of the present invention and conducting a batch fermentation at 37±2° C., for 48±2 hours; (H2) co-incubating 5% the bacterial broth and the liquid medium of the present invention and conducting a fed-batch fermentation at 37±2° C., for 48±2 hours; and (H3) co-incubating 5% the bacterial broth and the liquid medium of the present invention and conducting a fed-batch fermentation at 37±2° C., and then further conducting a fermentation on the solid medium of the present invention for 72±2 hours. In the present embodiment, fermentation products collected from the (H1), (H2) and (H3) are analyzed individually via the NBT assay. Furthermore, in the (H2), the original volume of the liquid medium is 100 ml, and 50 ml of fresh liquid medium (around 50% of the original volume of the liquid medium) are sequentially added to the liquid medium during the fed-batch fermentation.

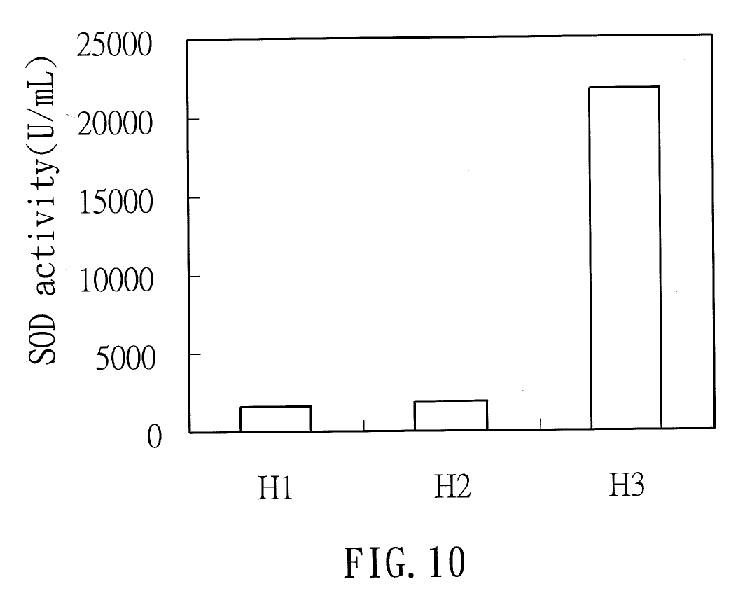

According to FIG. 10, it is shown that the fermentation products in the (H3) has SOD in higher activity among others, and therefore, it is believed that the method for increasing the activity of superoxide dismutase of the present invention is sufficient to promote the activity of SOD produced by Bacillus subtilis.

In summary, through the present invention, a method for increasing the activity of superoxide dismutase is provided by sequentially culturing a strain of Bacillus subtilis into a liquid medium and a solid medium to obtain a fermentation broth with highly active SOD. Therefore, it is sufficient to obtain high active of SOD from the Bacillus subtilis via a convenient and cost-saving process in the present invention.

Thus, since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A method for increasing the activity of superoxide dismutase comprising steps of:
liquid culture, by providing a strain of Bacillus subtilis and a liquid medium to carry out a fed-batch fermentation and to obtain a primary fermentation broth, wherein the liquid medium has 8 wt % to 10 wt % of maltose, 10 wt % to 15 wt % of soya powder and rest of water; and
solid culture, by culturing the primary fermentation broth in a solid medium, which has solid substrate and maltose in a ratio of 10:1 to 20:1, to obtain a secondary fermentation broth with superoxide dismutase (SOD) in high activity, wherein the solid substrate contains wheat germ and water.

2. The method for increasing the activity of superoxide dismutase as defined in claim 1, with the weight ratio between the Bacillus subtilis and the liquid medium being 3:97 to 5:95.

3. The method for increasing the activity of superoxide dismutase as defined in claim 1, with the temperature of the step of liquid culture being set up at 35° C. to 39° C. for 46 to 50 hours.

4. The method for increasing the activity of superoxide dismutase as defined in claim 1, wherein, in the step of liquid culture, fresh liquid medium is additionally added to maintain the pH of the liquid medium during the fed-batch fermentation.

5. The method for increasing the activity of superoxide dismutase as defined in claim 4, with volume of the fresh liquid medium additionally added during the fed-batch fermentation in the step of liquid culture being set up at 40% to 60% of the liquid medium.

6. The method for increasing the activity of superoxide dismutase as defined in claim 4, with the pH of the liquid medium being maintained at pH5.5 at lowest during the fed-batch fermentation.

7. The method for increasing the activity of superoxide dismutase as defined in claim 1, with the liquid medium further comprising 0.1% of sodium chloride.

8. The method for increasing the activity of superoxide dismutase as defined in claim 1, with the liquid medium further comprising 0.5% of tryptophane.

9. The method for increasing the activity of superoxide dismutase as defined in claim 1, with the liquid medium further comprising 10 ppm of vitamin B groups or vitamin B12.

10. The method for increasing the activity of superoxide dismutase as defined in claim 1, wherein the wheat germ of the solid substrate is cooked at 98° C. to 102° C. for 20 to 40 minutes before the culturing.

11. The method for increasing the activity of superoxide dismutase as defined in claim 10, wherein the solid substrate has cooked wheat germ and water in a weight ratio of 100:45 to 100:75.

12. The method for increasing the activity of superoxide dismutase as defined in claim 1, with the weight ratio of the primary fermentation broth being set up at 85 wt % to 95 wt %, and the weight ratio of the solid medium being set up at 5 wt % to 15 wt %.

13. The method for increasing the activity of superoxide dismutase as defined in claim 1, with the temperature of the step of solid culture being set up at 28° C. to 32° C. for 70 to 74 hours.
